(19) United States
(12) Patent Application Publication
(10) Pub. No.: US 2012/0027911 A1
(43) Pub. Date: Feb. 2, 2012

(54) PREPARATION OF SOY PROTEIN PRODUCT USING WATER EXTRACTION ("S803")

(76) Inventors: Kevin I. Segall, Winnipeg (CA); Martin Schweitzer, Winnipeg (CA); Brent E. Green, Winnipeg (CA); Sarah Medina, Winnipeg (CA); Brandy Gosnell, Winnipeg (CA)

(21) Appl. No.: 13/138,394

(22) PCT Filed: Feb. 11, 2010
(86) PCT No.: PCT/CA2010/000191

§ 371 Date: Oct. 19, 2011

Related U.S. Application Data
(60) Provisional application No. 61/202,260, filed on Feb. 11, 2009.
(60) Provisional application No. 61/272,288, filed on Sep. 8, 2009.

(51) Int. Cl. A23J 3/16; A23J 1/14; A23C 11/10; C07K 1/36; C07K 14/415

(52) U.S. Cl. 426/598; 530/378

(57) ABSTRACT

A soy protein product which is completely soluble and is capable of providing transparent and heat stable solutions at low and neutral pH values is produced by extracting a soy protein source material with water at low pH, subjecting the resulting aqueous soy protein solution to ultrafiltration and optional diafiltration to provide a concentrated and optionally diafiltered soy protein solution, which may be dried to provide the soy protein product. The soy protein product may be used for protein fortification of in particular, soft drinks and sports drinks, without precipitation of protein.

### REFERENCE TO RELATED APPLICATIONS

This application claims priority under 35 USC 119(e) from U.S. Provisional Patent Applications No. 61/202,260 filed Feb. 11, 2009 and No. 61/272,288 filed Sep. 8, 2009.

### FIELD OF INVENTION

The present invention is concerned with the preparation of soybean protein product.

### BACKGROUND TO THE INVENTION

In U.S. Provisional Patent Applications No. 61/107,112 filed Oct. 21, 2008 (7865-373), No. 61/193,457 filed Dec. 2, 2008 (7865-374), No. 61/202,070 filed Jan. 26, 2009 (7865-376), No. 61/202,553 filed Mar. 12, 2009 (7865-383), No. 61/213,717 filed Jul. 7, 2009 (7865-389), No. 61/272,241 filed Sep. 3, 2009 (7865-400) and U.S. patent application Ser. No. 12/603,087 filed Oct. 21, 2009, the disclosures of which are incorporated herein by reference, there is described the preparation of a soy protein product, preferably a soy protein isolate, which is completely soluble and is capable of providing transparent and heat stable solutions at low pH values. This soy protein product may be used for protein fortification of, in particular, soft drinks and sports drinks, as well as other acidic aqueous systems, without precipitation of protein. The soy protein product is produced by extracting a soy protein source with aqueous calcium chloride solution at natural pH, optionally diluting the resulting aqueous soy protein solution, adjusting the pH of the aqueous soy protein solution to a pH of about 1.5 to about 4.4, preferably about 2.0 to about 4.0, to produce an acidified clear soy protein solution, which may be optionally concentrated and/or diafiltered before drying.

### SUMMARY OF THE INVENTION

It has now been surprisingly found that a soy protein product of comparable properties may be formed by a procedure involving extraction of the soy protein source with water and without the necessity to use calcium chloride.

In one aspect of the present invention, a soy protein source material is extracted with water at low pH and the resulting aqueous soy protein solution is subjected to ultrafiltration and optional diafiltration to provide a concentrated and optionally diafiltered soy protein solution, which may be dried to provide the soy protein product.

The soy protein product provided herein, having a protein content of at least about 60 wt% (N×6.25) d.b., is soluble at acid pH values to provide transparent and heat stable aqueous solutions thereof. The soy protein product may be used for protein fortification of, in particular, soft drinks and sports drinks, as well as other aqueous systems without precipitation of protein. The soy protein product is preferably an isolate having a protein content of at least about 90 wt %, preferably at least about 100 wt % (N×6.25) d.b.

In accordance with one aspect of the present invention, there is provided a method of producing a soy protein product having a soy protein content of at least about 60 wt % on a dry weight basis (d.b.), which comprises:

(a) extracting a soy protein source with water at low pH to cause solubilization of soy protein from the protein source and to form an aqueous soy protein solution,

(b) separating the aqueous soy protein solution from residual soy protein source,

(c) concentrating the aqueous soy protein solution using a selective membrane technique,

(d) optionally diafiltering the concentrated soy protein solution, and

(e) optionally drying the concentrated soy protein solution.

The soy protein product preferably is an isolate having a protein content of at least about 90 wt %, preferably at least about 100 wt % (N×6.25) d.b.

While the present invention refers mainly to the production of soy protein isolates, it is contemplated that soy protein products of lesser purity may be provided having similar properties to the soy protein isolate. Such lesser purity products may have a protein concentration of at least about 60% by weight (N×6.25) d.b.

The novel soy protein product of the invention can be blended with powdered drinks for the formation of aqueous soft drinks or sports drinks by dissolving the same in water. Such blend may be a powdered beverage.

The soy protein product provided herein may be provided as an aqueous solution thereof having a high degree of clarity at acid pH values and which is heat stable at these pH values.

In another aspect of the present invention, there is provided an aqueous solution of the soy product provided herein which is heat stable at low pH. The aqueous solution may be a beverage, which may be a clear beverage in which the soy protein product is completely soluble and transparent or an opaque beverage in which the soy protein product does not increase the opacity. Aqueous solutions of the soy protein product also have excellent solubility and clarity at pH 7.

The soy protein product produced according to the process herein lacks the characteristic beany flavour of soy protein isolates and is suitable, not only for protein fortification of acid media, but may be used in wide variety of conventional applications of protein isolates, including but not limited to protein fortification of processed foods and beverages, emulsification of oils, as a body former in baked goods and foaming agent in products which entrap gases. In addition, the soy protein product may be formed into protein fibers, useful in meat analogs, and may be used as an egg white substitute or extender in food products where egg white is used as a binder. The soy protein product may also be used in nutritional supplements. Other uses of the soy protein product are in pet foods, animal feed and in industrial and cosmetic applications and in personal care products.

### GENERAL DESCRIPTION OF INVENTION

The initial step of the process of providing the soy protein product involves solubilizing soy protein from a soy protein source. The soy protein source may be soybeans or any soy product or by-product derived from the processing of soybeans including but not limited to soy meal, soy flakes, soy grits and soy flour. The soy protein source may be used in the full fat form, partially defatted form or fully defatted form. Where the soy protein source contains an appreciable amount of fat, an oil-removal step generally is required during the process. The soy protein recovered from the soy protein source may be the protein naturally occurring in soybean or the proteinaceous material may be a protein modified by genetic manipulation but possessing characteristic hydrophobic and polar properties of the natural protein.

Protein solubilization from the soy protein source material is effected herein using water at low pH. The extraction may be conducted at a pH of about 1.5 to about 3.6, preferably at a pH matching the pH of the product (for example, a beverage) in which the protein product is to be incorporated, such as a pH of about 2.6 to about 3.6. Generally, water is added to the soy protein source and then the pH is adjusted by the addition of any convenient food grade acid, usually hydrochloric acid or phosphoric acid. Where the soy protein product is intended for non-food uses, non-food-grade chemicals can be used.

In a batch process, the solubilization of the protein is effected at a temperature of from about 1° C. to about 100° C., preferably about 15° to about 35° C., preferably accompanied by agitation to decrease the solubilization time, which is usually about 1 to about 60 minutes. It is preferred to effect the solubilization to extract substantially as much protein from the soy protein source as is practicable, so as to provide an overall high product yield.

In a continuous process, the extraction of the soy protein from the soy protein source is carried out in any manner consistent with effecting a continuous extraction of soy protein from the soy protein source. In one embodiment, the soy protein source is continuously mixed with water and the mixture is conveyed through a pipe or conduit having a length and at a flow rate for a residence time sufficient to effect the desired extraction in accordance with the parameters described herein. In such a continuous procedure, the solubilization step is effected rapidly, in a time of up to about 10 minutes, preferably to effect solubilization to extract substantially as much protein from the soy protein source as is practicable. The solubilization in the continuous procedure is effected at temperatures between about 1° C. and about 100° C., preferably between about 15° C. and about 35° C.

The concentration of soy protein source in water during the solubilization step may vary widely. Typical concentration values are about 5 to about 15% w/v.

The protein extraction step may have the additional effect of solubilizing fats which may be present in the soy protein source, which then results in the fats being present in the aqueous phase.

The protein solution resulting from the extraction step generally has a protein concentration of about 5 to about 50 g/L, preferably about 10 to about 50 g/L.

An antioxidant may be present during the extraction step. The antioxidant may be any convenient antioxidant, such as sodium sulfite or ascorbic acid. The quantity of antioxidant employed may vary from about 0.01 to about 1 wt % of the solution, preferably about 0.05 wt %. The antioxidant serves to inhibit oxidation of any phenolics in the protein solution.

The aqueous phase resulting from the extraction step then may be separated from the residual soy protein source, in any convenient manner, such as by employing a decanter centrifuge, followed by disc centrifugation and/or filtration, to remove residual soy protein source material. The separated residual soy protein source may be dried for disposal. Alternatively, the separated residual soy protein source may be processed to recover some residual protein, such as by a conventional isoelectric precipitation procedure or any other convenient procedure to recover such residual protein.

Where the soy protein source contains significant quantities of fat, as described in U.S. Pat. Nos. 5,844,086 and 6,005,076, assigned to the assignee hereof and the disclosures of which are incorporated herein by reference, then the defatting steps described therein may be effected on the separated aqueous protein solution. Alternatively, defatting of the separated aqueous protein solution may be achieved by any other convenient procedure.

The aqueous soy protein solution may be treated with an adsorbent, such as powdered activated carbon or granulated activated carbon, to remove colour and/or odour compounds. Such adsorbent treatment may be carried out under any convenient conditions, generally at the ambient temperature of the separated aqueous protein solution. For powdered activated carbon, an amount of about 0.025% to about 5% w/v, preferably about 0.05% to about 2% w/v, is employed. The adsorbing agent may be removed from the soy protein solution by any convenient means, such as by filtration.

The clear aqueous acidified soy protein solution may be subjected to a heat treatment to inactivate heat labile anti-nutritional factors, such as trypsin inhibitors, present in the aqueous soy protein solution as a result of extraction from the soy protein source material during the extraction step. Such a heating step also provides the additional benefit of reducing the microbial load. Generally, the protein solution is heated to a temperature of about 70° to about 120° C., preferably about 85° to about 95° C., for about 10 seconds to about 60 minutes, preferably about 30 seconds to about 5 minutes. The heat treated soy protein solution then may be cooled for further processing as described below, to a temperature of about 2° to about 60° C., preferably about 20° to about 35° C.

If of adequate purity, the resulting aqueous soy protein solution may be directly dried to produce a soy protein product. To decrease the impurities content, the aqueous soy protein solution may be processed prior to drying.

The aqueous soy protein solution may be concentrated to increase the protein concentration thereof while maintaining the ionic strength thereof substantially constant. Such concentration generally is effected to provide a concentrated soy protein solution having a protein concentration of about 50 to about 400 g/L, preferably about 100 to about 250 g/L.

The concentration step may be effected in any convenient manner consistent with batch or continuous operation, such as by employing any convenient selective membrane technique, such as ultrafiltration or diafiltration, using membranes, such as hollow-fibre membranes or spiral-wound membranes, with a suitable molecular weight cut-off, such as about 3,000 to about 1,000,000 daltons, preferably about 5,000 to about 100,000 daltons, having regard to differing membrane materials and configurations, and, for continuous operation, dimensioned to permit the desired degree of concentration as the aqueous protein solution passes through the membranes.

As is well known, ultrafiltration and similar selective membrane techniques permit low molecular weight species to pass therethrough while preventing higher molecular weight species from so doing. The low molecular weight species extracted from the source material include carbohydrates, pigments, low molecular weight proteins and anti-nutritional factors, such as trypsin inhibitors, which are themselves low molecular weight proteins. The molecular weight cut-off of the membrane is usually chosen to ensure retention of a significant proportion of the protein in the solution, while permitting contaminants to pass through having regard to the different membrane materials and configurations.

The soy protein solution may be subjected to a diafiltration step, before or after complete concentration, using water. The water may be at its natural pH or at a pH equal to that of the protein solution being diafiltered or at any pH value in between. Such diafiltration may be effected using from about 2 to about 40 volumes of diafiltration solution, preferably about 5 to about 25 volumes of diafiltration solution. In the diafiltration operation, further quantities of contaminants are removed from the aqueous soy protein solution by passage through the membrane with the permeate. The diafiltration operation may be effected until no significant further quantities of contaminants or visible colour are present in the permeate or until the retentate has been sufficiently purified so as, when dried, to provide a product with the desired protein content, preferably an isolate with a protein content greater than 90 wt % (N×6.25) on a dry basis. Such diafiltration may be effected using the same membrane as for the concentration step. However, if desired, the diafiltration step may be effected using a separate membrane with a different molecular weight cut-off, such as a membrane having a molecular weight cut-off in the range of about 3,000 to about 1,000,000 Daltons, preferably about 5,000 to about 100,000 Daltons, having regard to different membrane materials and configuration.

The concentration step and the diafiltration step may be effected herein in such a manner that the soy protein product subsequently recovered by drying the concentrated and diafiltered retentate contains less than about 90 wt % protein (N×6.25) d.b., such as at least about 60 wt % protein (N×6.25) d.b. By partially concentrating and/or partially diafiltering the aqueous soy protein solution, it is possible to only partially remove contaminants. This protein solution may then be dried to provide a soy protein product with lower levels of purity. The soy protein product is still able to produce clear protein solutions under acidic conditions.

An antioxidant may be present in the diafiltration medium during at least part of the diafiltration step. The antioxidant may be any convenient antioxidant, such as sodium sulfite or ascorbic acid. The quantity of antioxidant employed in the diafiltration medium depends on the materials employed and may vary from about 0.01 to about 1 wt %, preferably about 0.05 wt %. The antioxidant serves to inhibit the oxidation of any phenolics present in the concentrated soy protein solution.

The concentration step and the optional diafiltration step may be effected at any convenient temperature, generally about 2° to about 60° C., preferably about 20° to about 35° C., and for the period of time to effect the desired degree of concentration and diafiltration. The temperature and other conditions used to some degree depend upon the membrane equipment used to effect the membrane processing and the desired protein concentration of the solution and the efficiency of removal of contaminants to the permeate.

There are two main trypsin inhibitors in soy, namely the Kunitz inhibitor, which is a heat-labile molecule with a molecular weight of approximately 21,000 Daltons, and the Bowman-Birk inhibitor, a more heat-stable molecule with a molecular weight of about 8,000 Daltons. The level of trypsin inhibitor activity in the final soy protein product can be controlled by manipulation of various process variables.

As noted above, heat treatment of the acidified aqueous soy protein solution may be used to inactivate heat-labile trypsin inhibitors. Such a heat treatment may also be applied to the concentrated and optionally diafiltered soy protein solution.

In addition, the concentration and/or diafiltration steps may be operated in a manner favorable for removal of trypsin inhibitors in the permeate along with the other contaminants. Removal of the trypsin inhibitors is promoted by using a membrane of larger pore size, such as about 30,000 to about 1,000,000 Daltons, operating the membrane at elevated temperatures, such as about 30° to about 60° C., and employing greater volumes of diafiltration medium, such as about 20 to about 40 volumes.

Acidifying and membrane processing the diluted protein solution at a lower pH, such as about 1.5 to about 3, may reduce the trypsin inhibitor activity relative to processing the solution at higher pH, such as about 3 to about 3.6. When the protein solution is concentrated and diafiltered at the low end of the pH range, it may be desired to raise the pH of the retentate prior to drying. The pH of the concentrated and diafiltered protein solution may be raised to the desired value, for example, about pH 3, by the addition of any convenient food grade alkali, such as sodium hydroxide.

Further, a reduction in trypsin inhibitor activity may be achieved by exposing soy materials to reducing agents that disrupt or rearrange the disulfide bonds of the inhibitors. Suitable reducing agents include sodium sulfite, cysteine and N-acetylcysteine.

The addition of such reducing agents may be effected at various stages of the overall process. The reducing agent may be added with the soy protein source material in the extraction step, may be added to the clarified aqueous soy protein solution following removal of residual soy protein source material, may be added to the concentrated protein solution before or after diafiltration or may be dry blended with the dried soy protein product. The addition of the reducing agent may be combined with a heat treatment step and the membrane processing steps, as described above.

If it is desired to retain active trypsin inhibitors in the concentrated protein solution, this can be achieved by eliminating or reducing the intensity of the heat treatment step, not utilizing reducing agents, operating the concentration and diafiltration steps at the higher end of the pH range, such as about 3 to about 3.6, utilizing a concentration and diafiltration membrane with a smaller pore size, operating the membrane at lower temperatures and employing fewer volumes of diafiltration medium.

The concentrated and optionally diafiltered protein solution may be subject to a further defatting operation, if required, as described in U.S. Pat. Nos. 5,844,086 and 6,005,076. Alternatively, defatting of the concentrated and optionally diafiltered protein solution may be achieved by any other convenient procedure.

The concentrated and optionally diafiltered clear aqueous protein solution may be treated with an adsorbent, such as powdered activated carbon or granulated activated carbon, to remove colour and/or odour compounds. Such adsorbent treatment may be carried out under any convenient conditions, generally at the ambient temperature of the concentrated protein solution. For powdered activated carbon, an amount of about 0.025% to about 5% w/v, preferably about 0.05% to about 2% w/v, is employed. The adsorbent may be removed from the soy protein solution by any convenient means, such as by filtration.

The concentrated and optionally diafiltered aqueous soy protein solution may be dried by any convenient technique, such as spray drying or freeze drying. A pasteurization step may be effected on the soy protein solution prior to drying. Such pasteurization may be effected under any desired pasteurization conditions. Generally, the concentrated and optionally diafiltered soy protein solution is heated to a temperature of about 55° to about 70° C., preferably about 60° to about 65° C., for about 30 seconds to about 60 minutes, preferably about 10 minutes to about 15 minutes. The pasteurized concentrated soy protein solution then may be cooled for drying, preferably to a temperature of about 15° to about 35° C.

The dry soy protein product has a protein content of at least about 60 wt %, preferably in excess of about 90 wt % protein, more preferably at least about 100 wt %, (N×6.25) d.b.

The soy protein product produced herein is soluble in an acidic aqueous environment, making the product ideal for incorporation into beverages, both carbonated and uncarbonated, to provide protein fortification thereto. Such beverages have a wide range of acidic pH values, ranging from about 2.5 to about 5. The soy protein product provided herein may be added to such beverages in any convenient quantity to provide protein fortification to such beverages, for example, at least about 5 g of soy protein per serving. The added soy protein product dissolves in the beverage and does not impair the clarity of the beverage, ever after thermal processing. The soy protein product may be blended with dried beverage prior to reconstitution of the beverage by dissolution in water. In some cases, modification to the normal formulation of the beverages to tolerate the composition of the invention may be necessary where components present in the beverage may adversely affect the ability of the composition of the invention to remain dissolved in the beverage. In addition, the soy protein product is highly soluble and produces solutions of excellent clarity at pH 7.

### EXAMPLES

### Example 1

This Example is an evaluation of the extractability of defatted, minimally heat processed soy flour with water or saline at low pH.

Defatted, minimally heat processed soy flour (10 g) was extracted with either water, 0.15 NaCl or 0.15M CaCl₂ (100 ml) with the pH of the extraction system adjusted to 3 with diluted HCl. Flour and solvent were combined, the pH adjusted and then the samples stirred for 30 minutes at room temperature using a magnetic stir bar and stir plate. The extract was separated from the spent meal by centrifugation at 10,200 g for 10 minutes and then further clarified by filtration with a 0.45 μm pore size syringe filter. The protein content of the filtrates was measured using a LECO FP528 Nitrogen Determinator and then the samples were diluted with an equal volume of water and observed for the presence of precipitate.

The extractability results are set forth in the following Table 1:

TABLE 1
Effect of extraction solvent on protein content of pH 3 extracts

| sample | % protein | extractability (%) |
|---|---|---|
| water | 3.38 | 62.2 |
| sodium chloride | 2.94 | 54.1 |
| calcium chloride | 3.79 | 69.8 |

As may be seen from the results of Table 1, the extractability was quite high for all the solvents, with the calcium chloride solution solubilizing the most protein. Extraction with water alone solubilized more protein than using 0.15M sodium chloride solution.

When the clarified extracts were diluted with water, the sodium chloride extract precipitated heavily, while the water and calcium chloride extracts stayed essentially clear.

### Example 2

This Example is an examination of the extractability of soy flour with water at various pH values and the clarity of the resulting extracts when acidified to pH 3.

Defatted, minimally heat processed soy flour (10 g) was extracted with reverse osmosis purified water (100 ml) for 30 minutes at room temperature using a magnetic stir bar/stir plate operated at constant speed. Timing of the 30 minutes for extraction started when stirring commenced. The pH of the extraction (water plus flour) was adjusted to 3, 5, 7, 9 or 11 with 6M HC1 or 6M NaOH immediately after the flour was entirely wetted (which occurred quite quickly) and monitored and corrected throughout the 30 minute extraction. After 30 minutes, the samples were centrifuged at 10,200 g for 10 minutes to separate extract from the spent meal. The extracts were then further clarified by filtration with a 0.45 um pore size syringe filter. The protein content of the filtered extracts was assessed using a LECO FP528 Nitrogen Determinator. The pH and clarity (A600) of the filtered extracts were also measured. A sample of filtered extract was diluted with one part reverse osmosis purified water and the pH and clarity of the diluted sample assessed. The full strength and diluted samples were then adjusted to pH 3 with 6M HCl or 6M NaOH as necessary and the clarity re-evaluated.

The effect of extraction pH on the extractability of the soy flour with water is set forth in the following Table 2:

TABLE 2
Effect of pH on the extractability of soy flour with water

| extraction pH | % protein in extract | extractability (%) |
|---|---|---|
| 3 | 2.43 | 45.4 |
| 5 | 0.70 | 13.1 |
| 7 | 4.05 | 75.7 |
| 9 | 4.28 | 80.0 |
| 11 | 5.18 | 96.8 |

As can be seen by the results in Table 2, significant extractabilities were obtained using water at alkaline pH. Although lower, the extractability obtained at pH 3 was a reasonable value.

The effect of acidification on the clarity of the full strength extract samples is set forth in the following Table 3:

TABLE 3
Effect of acidification on the clarity of full strength water extracts

| extraction pH | initial pH | initial A600 | adjusted pH | final A600 |
|---|---|---|---|---|
| 3 | 2.88 | 0.089 | 2.96 | 0.095 |
| 5 | 4.99 | 0.007 | 3.05 | 2.58 |
| 7 | 6.96 | 0.155 | 3.04 | >3.0 |
| 9 | 8.87 | 0.222 | 3.02 | >3.0 |
| 11 | 10.92 | 0.173 | 2.95 | >3.0 |

As can be seen in the results of Table 3, the sample extracted at pH 3 was the only sample that remained clear after pH adjustment.

The effect of acidification on the clarity of the diluted extract samples is set forth in the following Table 4:

TABLE 4
Effect of acidification on the clarity of diluted water extracts

| extraction pH | initial pH | initial A600 | adjusted pH | final A600 |
|---|---|---|---|---|
| 3 | 2.97 | 0.222 | — | — |
| 5 | 5.06 | 0.001 | 2.96 | 2.53 |
| 7 | 6.97 | 0.080 | 3.02 | >3.0 |
| 9 | 8.80 | 0.129 | 2.97 | 0.334 |
| 11 | 10.86 | 0.062 | 2.96 | 1.55 |

As can be seen from the results of Table 4, the sample extracted at pH 3 and then diluted was the clearest of those evaluated.

### Example 3

This Example was conducted to determine if a low pH water extract of soy flour would stay clear when concentrated and diafiltered and also re-hydrate clear after drying.

80 g of defatted, minimally heat processed soy flour was added to 800 ml of reverse osmosis purified water at ambient temperature and agitated for 30 minutes to provide an aqueous protein solution. Immediately after the flour was dispersed in the water, the pH of the system was adjusted to 3 by the addition of diluted HCl. The pH was monitored and corrected to 3 periodically over the course of the 30 minute extraction.

The residual soy flour was removed and the resulting protein solution was clarified by centrifugation and filtration to produce 475 ml of filtered protein solution having a protein content of 1.86% by weight.

The filtered protein solution was reduced in volume to 42 ml by concentration on a polyethersulfone (PES) membrane having a molecular weight cut-off of 10,000 Daltons. An aliquot of 40 ml of concentrated protein solution was diafiltered with 80 ml of reverse osmosis purified water. The resulting diafiltered, concentrated protein solution had a protein content of 15.42% by weight and represented a yield of 69.2 wt % of the initial filtered protein solution. The diafiltered, concentrated protein solution was then dried to yield a product found to have a protein content of 90.89% (N×6.25) w.b. The product was termed 5803.

A 3.2 wt % protein solution of 5803 in water was prepared and the colour and clarity assessed using a HunterLab Color Quest XE instrument operated in transmission mode.

The colour and clarity values are set forth in the following Table 5:

TABLE 5
HunterLab scores for 3.2% protein solution of S803

| sample | L* | a* | b* | haze (%) |
|---|---|---|---|---|
| S803 | 96.97 | −1.39 | 10.87 | 17.6 |

As may be seen from Table 5, the colour of the 5803 solution was very light and the haze level was quite low.

### Example 4

In this Example, the heat stability of the S803 product, produced according to the procedure of Example 3, was assessed.

A 2% w/v protein solution of 5803 in water was produced. The pH of the solution was determined with a pH meter and the clarity of the solution was assessed by haze measurement with the HunterLab Color Quest XE instrument. The solution was then heated to 95° C., held at this temperature for 30 seconds and then immediately cooled to room temperature in an ice bath. The clarity of the heat treated solution was then measured.

The pH of the 5803 solution was 2.91. The clarity of the protein solution before and after heating is set forth in the following Table 6:

TABLE 6
Effect of heat treatment on clarity of S803 solution

| sample | haze (%) |
|---|---|
| before heating | 53.8 |
| after heating | 32.4 |

As can be seen from Table 6, the clarity of the 2% solution of 5803 was inferior to that of the 3.2% solution prepared in Example 3. The reason for this was unknown. In any case, when the 2% protein solution was heat treated the haze level in the sample was reduced. Therefore, heat treatment did not impair the clarity.

### Example 5

In this Example, the production of 5803 was scaled up from benchtop to pilot plant scale.

‘a’ kg of defatted, minimally heat processed soy flour was added to ‘b’ L of reverse osmosis purified water at ambient temperature and agitated for 30 minutes to provide an aqueous protein solution. Immediately after the flour was dispersed in the water, the pH of the system was adjusted to 3 by the addition of dilute HCl. The pH was monitored and corrected to 3 periodically over the course of the 30 minute extraction. The residual soy flour was removed and the resulting protein solution was clarified by centrifugation and filtration to produce ‘c’ L of filtered protein solution having a protein content of ‘d’% by weight.

The filtered protein solution was reduced in volume to ‘e’ L by concentration on a T membrane having a molecular weight cut-off of ‘g’ Daltons. An aliquot of ‘h’ L of concentrated protein solution with a protein content of ‘i’% by weight and representing a yield of T wt % of the initial filtered protein solution was dried to yield a product found to have a protein content of ‘k’% (N×6.25) d.b. The product was termed ‘1’ S803-02. The remaining ‘m’ L of concentrated protein solution was diafiltered with ‘n’ L of reverse osmosis purified water ‘o’. The resulting diafiltered, concentrated protein solution had a protein content of ‘p’% by weight and represented a yield of ‘q’ wt % of the initial filtered protein solution. The diafiltered, concentrated protein solution was then dried to yield a product found to have a protein content of ‘r’% (N×6.25) d.b. The product was termed ‘1’ 5803.

The parameters ‘a’ to ‘r’ for two runs are set forth in the following Table 7:

TABLE 7
Parameters for the runs to produce S803

| l | S005-L16-08A | S005-A20-09A |
|---|---|---|
| a | 20 | 20 |
| b | 200 | 200 |
| c | 170 | 210 |
| d | 0.71 | 0.91 |
| e | 18.46 | 25 |
| f | PVDF | PVDF |
| g | 5,000 | 5,000 |
| h | 2 | 0 |
| i | 6.21 | n/a |
| j | 9.9 | n/a |
| k | 95.96 | n/a |
| m | 16.46 | 25 |
| n | 34 | 50 |
| o | adjusted to pH 3 with diluted HCl | at natural pH |
| p | 6.29 | 8.69 |
| q | 86.0 | 93.2 |
| r | 94.63 | 98.36 |

n/a = not applicable

3.2% w/v protein solutions of S005-L16-08A 5803, S803-02 and S005-A20-09A 5803 were prepared in water and the colour and clarity assessed using a HunterLab Color Quest XE instrument operated in transmission mode. The pH was also measured with a pH meter.

The pH, colour and clarity values are set forth in the following Table 8:

TABLE 8
pH and HunterLab scores for 3.2% protein solutions of S005-L16-08A
S803, S803-02 and S005-A20-09A S803

| sample | pH | L* | a* | b* | haze (%) |
|---|---|---|---|---|---|
| S005-L16-08A S803 | 3.37 | 96.09 | 0.24 | 9.17 | 2.9 |
| S005-L16-08A S803-02 | 3.52 | 96.11 | −0.90 | 10.29 | 8.9 |
| S005-A20-09A S803 | 3.13 | 95.98 | −0.65 | 9.98 | 10.2 |

As may be seen from Table 8, the colours of the 5803 solutions were very light and the haze levels were low.

The colour of the dry powders was also assessed with the HunterLab Color Quest XE instrument in reflectance mode. The colour values are set forth in the following Table 9:

TABLE 9
HunterLab scores for S005-L16-08A S803, S803-02 and S005-A20-09A
S803 dry powders

| sample | L* | a* | b* |
|---|---|---|---|
| S005-L16-08A S803 | 87.88 | 0.02 | 6.90 |
| S005-L16-08A S803-02 | 88.84 | −0.28 | 7.83 |
| S005-A20-09A S803 | 87.07 | −0.03 | 8.47 |

As may be seen from Table 9, all dry products were very light in colour.

### Example 6

This Example contains an evaluation of the heat stability in water of the soy protein isolates produced by the method of Example 5 (S803).

2% w/v protein solutions of S005-L16-08A 5803 and S005-A20-09A S803 were produced in water and the pH adjusted to 3. The clarity of these solutions was assessed by haze measurement with the HunterLab Color Quest XE instrument in transmission mode. The solutions were then heated to 95° C., held at this temperature for 30 seconds and then immediately cooled to room temperature in an ice bath. The clarity of the heat treated solutions was then measured again.

The clarity of the protein solutions before and after heating is set forth in the following Table 10:

TABLE 10
Effect of heat treatment on clarity of S005-L16-08A S803 and S005-A20-
09A S803 solutions

| sample | Haze (%) before heating | Haze (%) after heating |
|---|---|---|
| S005-L16-08A S803 | 5.0 | 1.7 |
| S005-A20-09A S803 | 16.2 | 13.5 |

As can be seen from the results in Table 10, the clarity of these 2% solutions of 5803 prepared at pilot scale as described in Example 5 was much better than the clarity of the 2% solution of S803 prepared at laboratory scale as described in Example 3. It is unknown why this difference occurred. As was the case in Example 4, the solutions of 5803 were found to be heat stable with the heat treatment appearing to improve the clarity.

### Example 7

This Example contains an evaluation of the solubility in water of the soy protein isolates produced by the method of Example 5 (S803). Solubility was tested based on protein solubility (termed protein method, a modified version of the procedure of Mon et al., J. Food Sci. 50:1715-1718) and total product solubility (termed pellet method).

Sufficient protein powder to supply 0.5 g of protein was weighed into a beaker and then a small amount of reverse osmosis (RO) purified water was added and the mixture stirred until a smooth paste formed. Additional water was then added to bring the volume to approximately 45 ml. The contents of the beaker were then slowly stirred for 60 minutes using a magnetic stirrer. The pH was determined immediately after dispersing the protein and was adjusted to the appropriate level (2, 3, 4, 5, 6 or 7) with diluted NaOH or HCl. A sample was also prepared at natural pH. For the pH adjusted samples, the pH was measured and corrected two times during the 60 minutes stirring. After the 60 minutes of stirring, the samples were made up to 50 ml total volume with RO water, yielding a 1% w/v protein dispersion. The protein content of the dispersions was measured using a LECO FP528 Nitrogen Determinator. Aliquots (20 ml) of the dispersions were then transferred to pre-weighed centrifuge tubes that had been dried overnight in a 100° C. oven then cooled in a desiccator and the tubes capped. The samples were centrifuged at 7800 g for 10 minutes, which sedimented insoluble material and yielded a clear supernatant. The protein content of the supernatant was measured by LECO analysis and then the supernatant and the tube lids were discarded and the pellet material dried overnight in an oven set at 100° C. The next morning the tubes were transferred to a desiccator and allowed to cool. The weight of dry pellet material was recorded. The dry weight of the initial protein powder was calculated by multiplying the weight of powder used by a factor of ((100-moisture content of the powder (%))/100). Solubility of the product was then calculated two different ways:

1) Solubility (protein method) (%)=(% protein in supernatant/% protein in initial dispersion)×100

2) Solubility (pellet method) (%)=(1-(weight dry insoluble pellet material/((weight of 20 ml of dispersion/weight of 50 ml of dispersion) x initial weight dry protein powder)))×100

The natural pH values of the protein isolates produced in Example 5 in water (1% protein) are shown in Table 11:

TABLE 11
Natural pH of solutions prepared in water at 1% protein

| Batch | Product | Natural pH |
|---|---|---|
| S005-L16-08A | S803 | 3.36 |
| S005-A20-09A | S803 | 3.14 |

The solubility results obtained are set forth in the following Tables 12 and 13:

TABLE 12
Solubility of S803 at different pH values based on protein method

| | | Solubility (protein method) (%) | | | | | | |
|---|---|---|---|---|---|---|---|---|
| Batch | Product | pH 2 | pH 3 | pH 4 | pH 5 | pH 6 | pH 7 | Nat. pH |
| S005- | S803 | 92.6 | 95.7 | 66.3 | 16.1 | 84.4 | 100 | 92.9 |
| L16-08A | | | | | | | | |
| S005- | S803 | 90.3 | 95.7 | 29.3 | 10.1 | 90.1 | 86.9 | 91.8 |
| A20-09A | | | | | | | | |

TABLE 13
Solubility of S803 at different pH values based on pellet method

| | | Solubility (pellet method) (%) | | | | | | |
|---|---|---|---|---|---|---|---|---|
| Batch | Product | pH 2 | pH 3 | pH 4 | pH 5 | pH 6 | pH 7 | Nat. pH |
| S005- | S803 | 97.2 | 97.1 | 67.5 | 22.5 | 84.1 | 97.8 | 97.1 |
| L16-08A | | | | | | | | |
| S005- | S803 | 97.1 | 96.9 | 36.3 | 26.5 | 88.1 | 97.5 | 97.2 |
| A20-09A | | | | | | | | |

As can be seen from the results of Tables 12 and 13, the 5803 products were extremely soluble at pH values of 2, 3 and 7 and at the natural pH.

### Example 8

This Example contains an evaluation of the clarity in water of the soy protein isolates produced by the method of Example 5 (S803).

The clarity of the 1% w/v protein dispersions prepared as described in Example 7 was assessed by measuring the absorbance at 600 nm, with a lower absorbance score indicating greater clarity. Analysis of the samples on a HunterLab Color Quest XE instrument in transmission mode also provided a percentage haze reading, another measure of clarity.

The clarity results are set forth in the following Tables 14 and 15:

TABLE 14
Clarity of S803 solutions at different
pH values as assessed by A600

| | | A600 | | | | | | |
|---|---|---|---|---|---|---|---|---|
| Batch | Product | pH 2 | pH 3 | pH 4 | pH 5 | pH 6 | pH 7 | Nat. pH |
| S005- | S803 | 0.013 | 0.026 | >3.0 | >3.0 | 1.077 | 0.021 | 0.036 |
| L16-08A | | | | | | | | |
| S005- | S803 | 0.031 | 0.070 | >3.0 | >3.0 | 0.704 | 0.034 | 0.065 |
| A20-09A | | | | | | | | |

TABLE 15
Clarity of S803 solutions at different pH
values as assessed by HunterLab analysis

| | | HunterLab haze reading (%) | | | | | | |
|---|---|---|---|---|---|---|---|---|
| Batch | Product | pH 2 | pH 3 | pH 4 | pH 5 | pH 6 | pH 7 | Nat. pH |
| S005- | S803 | 1.8 | 4.6 | 95.7 | 96.1 | 83.2 | 1.7 | 4.9 |
| L16-08A | | | | | | | | |
| S005- | S803 | 1.4 | 9.5 | 95.4 | 95.7 | 68.2 | 0.0 | 8.6 |
| A20-09A | | | | | | | | |

As can be seen from the results of Tables 14 and 15, solutions of S803 exhibited excellent clarity at pH values of 2, 3 and 7 and at the natural pH.

### Example 9

This Example contains an evaluation of the solubility in a soft drink (Sprite) and sports drink (Orange Gatorade) of the soy protein isolate produced by the method of Example 5 (S803). The solubility was determined with the protein added to the beverages with no pH correction and again with the pH of the protein fortified beverages adjusted to the level of the original beverages.

When the solubility was assessed with no pH correction, a sufficient amount of protein powder to supply 1 g of protein was weighed into a beaker and a small amount of beverage was added and stirred until a smooth paste formed. Additional beverage was added to bring the volume to 50 ml, and then the solutions were stirred slowly on a magnetic stirrer for 60 minutes to yield a 2% protein w/v dispersion. The protein content of the samples was analyzed using a LECO FP528 Nitrogen Determinator then an aliquot of the protein containing beverages was centrifuged at 7800 g for 10 minutes and the protein content of the supernatant measured.

Solubility (%)=(% protein in supernatant/% protein in initial dispersion)×100

When the solubility was assessed with pH correction, the pH of the soft drink (Sprite) (3.39) and sports drink (Orange Gatorade) (3.19) without protein was measured. A sufficient amount of protein powder to supply 1 g of protein was weighed into a beaker and a small amount of beverage was added and stirred until a smooth paste formed. Additional beverage was added to bring the volume to approximately 45 ml, and then the solutions were stirred slowly on a magnetic stirrer for 60 minutes. The pH of the protein containing beverages was measured and then adjusted to the original no-protein pH with HCl or NaOH as necessary. The total volume of each solution was then brought to 50 ml with additional beverage, yielding a 2% protein w/v dispersion. The protein content of the samples was analyzed using a LECO FP528 Nitrogen Determinator then an aliquot of the protein containing beverages was centrifuged at 7800 g for 10 minutes and the protein content of the supernatant measured.

Solubility (%)=(% protein in supernatant/% protein in initial dispersion)×100

The results obtained are set forth in the following Table 16:

TABLE 16
Solubility of S803 in Sprite and Orange Gatorade

| | | no pH correction | | pH correction | |
|---|---|---|---|---|---|
| | | | Solubility | | Solubility |
| | | Solubility | (%) | Solubility | (%) |
| | | (%) | in Orange | (%) in | in Orange |
| Batch | Product | in Sprite | Gatorade | Sprite | Gatorade |
| S005-L16-08A | S803 | 97.7 | 100 | 100 | 100 |
| S005-A20-09A | S803 | 100 | 100 | 100 | 100 |

As can be seen from the results of Table 16, the 5803 was extremely soluble in the Sprite and the Orange Gatorade. As S803 is an acidified product, protein addition had little effect on beverage pH.

### Example 10

This Example contains an evaluation of the clarity in a soft drink and sports drink of the soy protein isolate produced by the method of Example 5 (S803).

The clarity of the 2% w/v protein dispersions prepared in soft drink (Sprite) and sports drink (Orange Gatorade) in Example 9 were assessed using the methods described in Example 8. For the absorbance measurements at 600 nm, the spectrophotometer was blanked with the appropriate beverage before the measurement was performed.

The results obtained are set forth in the following Tables 17 and 18:

TABLE 17
Clarity (A600) of S803 in Sprite and Orange Gatorade

| | | no pH correction | | pH correction | |
|---|---|---|---|---|---|
| | | | A600 in | | A600 in |
| | | A600 in | Orange | A600 in | Orange |
| Batch | Product | Sprite | Gatorade | Sprite | Gatorade |
| S005-L16-08A | S803 | 0.062 | 0.220 | 0.067 | 0.484 |
| S005-A20-09A | S803 | 0.132 | 0.101 | 0.099 | 0.115 |

TABLE 18
HunterLab haze readings for S803 in Sprite and Orange Gatorade

| | | | | pH correction | |
|---|---|---|---|---|---|
| | | no pH correction | | | haze |
| | | haze (%) | haze (%) in | haze | (%) in |
| | | in | Orange | (%) in | Orange |
| Batch | Product | Sprite | Gatorade | Sprite | Gatorade |
| no protein | | 0.0 | 44.0 | 0.0 | 44.0 |
| S005-L16-08A | S803 | 10.7 | 65.7 | 17.0 | 81.9 |
| S005-A20-09A | S803 | 24.8 | 59.2 | 14.4 | 52.3 |

As can be seen from the results of Tables 17 and 18, the S005-L16-08A S803 increased the haze in Orange Gatorade much more than the S005-A20-09A S803 did. The reason for this was unknown. When both S803 products were put into Sprite, the beverage was substantially clear or perhaps slightly hazy.

### SUMMARY OF THE DISCLOSURE

In summary of this disclosure, the present invention provides a method of producing a soy protein product which is soluble in acid media, based on water extraction of a soy protein source material. Modifications are possible within the scope of this invention.

What we claim is:

1. A process of preparing a soy protein product having a soy protein content of at least about 60 wt % (N×6.25) on a dry weight basis, which comprises:
(a) extracting a soy protein source with water at low pH to cause solubilization of soy protein from the protein source and to form an aqueous soy protein solution,
(b) separating the aqueous soy protein solution from residual soy protein source,
(c) concentrating the aqueous soy protein solution using a selective membrane technique,
(d) optionally diafiltering the concentrated soy protein solution, and
(e) optionally drying the concentrated soy protein solution.

2. The process of claim 1 wherein said water has a pH of about 1.5 to about 3.6.

3. The process of claim 2 wherein the pH is about 2.6 to about 3.6.

4. The process of claim 1 wherein said extraction step is effected at a temperature of about 15° C. to about 35° C.

5. The process of claim 1 wherein said aqueous soy protein solution has a protein concentration of about 5 to about 50 g/L.

6. The process of claim 5 wherein said aqueous soy protein solution has a protein concentration of about 10 to about 50 g/L.

7. The process of claim 1 wherein said water contains an antioxidant.

8. The process of claim 1 wherein said aqueous soy protein solution is treated with an adsorbent to remove colour and/or odour compounds from the aqueous soy protein solution.

9. The process of claim 1 wherein said aqueous soy protein solution is subjected to a heat treatment to inactivate heat labile anti-nutritional factors.

10. The process of claim 9 wherein the anti-nutritional factors are heat-labile trypsin inhibitors.

11. The process of claim 9 wherein the heat treatment step also pasteurizes the acidified clear aqueous protein solution.

12. The process of claim 9 wherein said heat treatment step is effected at a temperature of about 70° to about 120° C. for about 10 seconds to about 60 minutes.

13. The process of claim 12 wherein said heat treatment step is effected at a temperature of about 85° to about 95° C. for about 30 seconds to about 5 minutes.

14. The process of claim 9 wherein the heat-treated soy protein solution is cooled to a temperature of about 2° to about 60° C. for further processing.

15. The process of claim 14 wherein the heat-treated soy protein solution is cooled to a temperature of about 20° to about 35° C. for further processing.

16. The process of claim 1 wherein the aqueous soy protein solution is concentrated to a protein concentration of about 50 to about 400 g/L.

17. The process of claim 16 wherein the aqueous protein solution is concentrated to a protein concentration of about 100 to about 250 g/L.

18. The process of claim 1 wherein the aqueous soy protein solution is concentrated using a membrane having a molecular weight cut-off of about 3,000 to about 1,000,000 Daltons.

19. The process of claim 18 wherein the aqueous soy protein solution is concentrated using a membrane having a molecular weight cut-off of about 5,000 to about 100,000 Daltons.

20. The process of claim 1 wherein the optional diafiltration step is effected using water or acidified water on the soy protein solution before or after complete concentration thereof.

21. The process of claim 20 wherein the optional diafiltration step is effected using from about 2 to about 40 volumes of diafiltration solution.

22. The process of claim 21 wherein the optional diafiltration step is effected using from about 5 to about 25 volumes of diafiltration solution.

23. The process of claim 20 wherein said diafiltration step is effected using a membrane having a molecular weight cut-off of about 3,000 to about 1,000,000 Daltons.

24. The process of claim 23 wherein said diafiltration step is effected using a membrane having a molecular weight cut-off of about 5,000 to about 100,000 Daltons.

25. The process of claim 20 wherein an antioxidant is present during at least part of the diafiltration step.

26. The process of claim 20 wherein said optional diafiltration step is effected until no significant further quantities of contaminants or visible colour are present in the permeate.

27. The process of claim 20 wherein said optional diafiltration is effected until the retentate has been sufficiently purified so as, when dried, to provide a soy protein product with a protein content of at least about 60 wt % (N×6.25) d.b.

28. The process of claim 27 wherein said optional diafiltration is effected until the retentate has been sufficiently purified so as, when dried, to provide a soy protein isolate with a protein content of at least about 90 wt % (N×6.25) d.b.

29. The process of claim 28 wherein said optional diafiltration is effected until the retentate has been sufficiently purified so as, when dried, to provide a soy protein isolate with a protein content of at least about 100 wt % (N×6.25) d.b.

30. The method of claim 1 wherein said concentration step and optional diafiltration step are carried out at a temperature of about 2° to about 60° C.

31. The method of claim 30 wherein said temperature is about 20° to about 35° C.

32. The process of claim 1 wherein the concentration and/or optional diafiltration step are operated in a manner favourable to the removal of trypsin inhibitors.

33. The process of claim 1 wherein the concentrated and optionally diafiltered soy protein solution is treated with an adsorbent to remove colour and/or odour compounds prior to said drying step.

34. The process of claim 1 wherein the concentrated and optionally diafiltered soy protein solution is pasteurized prior to drying.

35. The method of claim 34 wherein said pasteurization step is effected at a temperature of about 55° to about 70° C. for about 30 seconds to about 60 minutes.

36. The method of claim 35 wherein said pasteurization step is effected at a temperature of about 60° to about 65° C. for about 10 to about 15 minutes.

37. The method of claim 34 wherein said pasteurized, concentrated and optionally diafiltered soy protein solution is cooled to a temperature of about 15° C. to about 35° C. for drying or further processing.

38. The process of claim 1 wherein a reducing agent is present during the extraction step to disrupt or rearrange the disulfide bonds of trypsin inhibitors to achieve a reduction in trypsin inhibitor activity.

39. The process of claim 1 wherein a reducing agent is present during the concentration and/or optional diafiltration step to disrupt or rearrange the disulfide bonds of trypsin inhibitors to achieve a reduction in trypsin inhibitor activity.

40. The process of claim 1 wherein a reducing agent is added to the concentrated and optionally diafiltered soy protein solution prior to drying and/or the dried soy protein product to disrupt or rearrange the disulfide bonds of trypsin inhibitors to achieve a reduction in trypsin inhibitor activity.

41. The process of claim 1 wherein the concentrated and optionally diafiltered soy protein solution is dried to provide a soy protein product having a protein content of about 60 to about 90 wt % (N×6.25) d.b.

42. The process of claim 1 wherein the concentrated and optionally diafiltered soy protein solution is dried to provide a soy protein isolate having a protein content of at least about 90 wt % (N×6.25) d.b.

43. The process of claim 1 wherein the concentrated and optionally diafiltered soy protein solution is dried to provide a soy protein isolate having a protein content of at least about 100 wt % (N×6.25) d.b.

44. A soy protein product produced by the process of claim 1.

45. An acidic solution having dissolved therein the soy protein product of claim 44.

46. The aqueous solution of claim 45 which is a beverage.

47. The soy protein product of claim 44 which is blended with water-soluble powdered materials for the production of aqueous solutions of the blend.

48. The blend of claim 47 which is a powdered beverage.

49. A neutral solution having dissolved therein the soy protein product of claim 44.
